(19) United States
(12) Patent Application Publication
(10) Pub. No.: US 2013/0059916 A1
(43) Pub. Date: Mar. 7, 2013

(54) BIGUANIDE COMPOUNDS AND ITS USE FOR TREATING CANCER

(71) Applicants: Stephane Rocchi, Nice (FR); Vincent Aroles, Lyon (FR); Robert Ballotti, Nice (FR); Tijana Tomic, Nice (FR)

(72) Inventors: Stephane Rocchi, Nice (FR); Vincent Aroles, Lyon (FR); Robert Ballotti, Nice (FR); Tijana Tomic, Nice (FR)

(21) Appl. No.: 13/696,347

(22) PCT Filed: May 6, 2010
(86) PCT No.: PCT/EP11/02268

§ 371 Date: Nov. 6, 2012

(30) Foreign Application Priority Data
May 26, 2010 (EP) 10005432.9

(51) Int. Cl. A61K 31/155; A61P 35/02; A61P 35/00

(52) U.S. Cl. 514/635

(57) ABSTRACT

The invention relates to a compound of formula (I), wherein the radicals R¹ to R² have the meaning according to claim 1, and/or physiologically acceptable salts thereof for the use for the prevention and/or treatment of cancer.

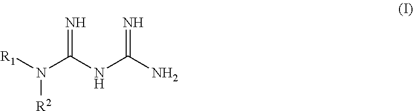

The invention relates to biguanides derivatives with the formula I

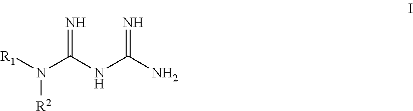

wherein the radicals R¹ and R² have the meaning according to claim 1, and/or physiologically acceptable salts thereof, for the use of these compounds for the prevention and/or treatment of cancer.

### BACKGROUND OF THE INVENTION

The biguanides family has been known for years for their therapeutic activity in the field of metabolic syndrome more particulary for the treatment of non insulin dependant diabetes (type II diabetes, NIDDM).

The metabolic syndrome refers to the clustering of cardiovascular risk factors that include diabetes, obesity, dyslipidaemia and hypertension. Insulin resistance and visceral obesity have been recognized as the most important pathogenic factors. Insulin resistance could be defined as the inability of insulin to produce its numerous actions, in spite of the unimpaired secretion from the beta cells. Metabolic abnormalities result from the interaction between the effects of insulin resistance located primarily in the muscle and adipose tissue and the adverse impact of the compensatory hyperinsulinaemia on tissues that remain normally insulin-sensitive.

The clinical heterogeneity of the syndrome also known as syndrome X or Insulin Resistance Syndrome can be explained by its significant impact on glucose, fat and protein metabolism, cellular growth and differentiation, and endothelial function. Visceral fat represents a metabolically active organ, strongly related to insulin sensitivity.

Metabolic syndrome increases the risk of diabetes of type II anywhere from 9-30 times over the normal population. As to the risk of heart disease, studies vary, but the metabolic syndrome appears to increase the risk 2-4 times that of the normal population.

Metformin (compound of formula I, wherein R¹, R²=methyl) as antidiabetic compound was included in a clinical study and its positive effect in metabolic syndrome and associated pathologies (hypertension, dyslipidemia, obesity). This metabolic syndrome was defined according to criteria from National Cholesterol Education Program (JAMA 2001, 2486) with criteria on waist circonference, serum triglyceride level, Hight Density Lipoprotein (HDL) cholesterol level, blood pressure and fasting plasma glucose level.

The metformin (dimethylbiguanide hydrochloride: R¹═R²═CH₃) trade under the brand Glucophage has been used for the treatment of non insulin dependant diabetes mellitus (NIDDM) also named diabetes of type II in order to decrease hyperglycemia.

Metformin decreases hyperglycemia by reducing the hepatic gluconeogenesis and decreasing the insulin resistance, improving entering of glucose into cells; this compound has no effect on the secretion of insulin by contrast with the sulfonylureas hypoglycemic drugs which are insulinosecretors.

Phenformin (phenethylbiguanides R¹=beta-phenylethyl, R²═H) also launched as hypoglycemic drug was discontinued in USA in 1977 due to its lactic acidosis. In case of use of metformin, patients have a better and bearable management of the lactic acid rate avoiding adverse effect.

The management of glucose homeostasis has a key role in the cells funtioning of the normal patient.

A number of epidemiologic studies have identified an increased risk of development of cancer in people with diabetes of type II (NIDDM), the association seems to be mediated with people having metabolic syndrome.

There is also evidence that impaired glucose tolerance may lead to an increase risk of cancer. Insulin is a growth-promoting hormone with mitogenic effect and it has been suggested that hyperinsulinemia combined with insulin resistance might promote carcinogenesis.

Observational study shows the risk of developing cancer over 5 years with insulin. We found that patients on insulin were twice as likely to develop colon carcinomas. With pancreatic cancer there is a major difference between the 2—patients on insulin alone had 4.5 times the risk.

AMPK is well established as a sensor and regulator of cellular energy homeostasis (Hardie D. G. and Hawley S. A; “AMP-activated protein kinase: the energy charge hypothesis revisited” Bioassays, 23, 1112, (2001), Kemp B. E. et al. “AMP-activated protein kinase, super metabolic regulator”.

WO 2005/023202 A2 from Beth Israel Deaconess Medical Center; Dana-Farber Cancer Institute relates to screening for agents that modulate the activity of LKB1 or AMPK protein. The application describes a method for identifying compounds that are useful in the treatment of diabetes or in the treatment of cancer.

Present invention relates to biguanide compounds of formula I that do not activate AMPK protein and where AMPK pathway is not involved in the therapeutic effect. The present invention relates to the use of biguanide compounds of formula I for prevention and/or the treatment of cancer.

In contrast to the disclosure in WO 2005/023202, metformin does not increase AMPK activity in subject to be treated.

A second embodiment is the use of biguanides of formula I for the prevention and/or treatment of cancer, wherein cancer is selected from tumors of squamous epithelium, the bladder, the stomach, the kidneys, of head and neck, the oesophagus, the cervix, the thyroid, the intestine, the liver, the brain, the prostate, the urogenital tract, the lymphatic system, the stomach, the bones, the skin, the larynx, the lung.

A prefered embodiment is the use of biguanides formula I for the prevention and/or treatment of cancer, wherein cancer is selected from the group monocytic leukaemia, lung adenocarcinoma, small-cell lung carcinomas, pancreatic cancer, glioblastomas, breast carcinoma, lung adenocarcinoma, small-cell lung carcinomas, pancreatic cancer, colon carcinoma, bones malignant tumor like osteosarcoma, chondrosarcoma, Ewing's sarcoma, melanoma.

A third embodiment is focussed on the use of biguanides of formula I for the prevention and/or the treatment of cancer of the skin and more particular on cancer named melanoma.

A forth embodiment is the use of biguanides derivatives of formula I for the prevention and/or treatment of cancer, which comprises the administration of one or more compounds of the formula I to a patient in need of such an administration, and where the patient also suffers from a disease selected from diabetes, dyslipidemia, hypertension, retinopathy, nephropathy, neuropathy, obesity connected with the Metabolic Syndrome.

A fifth embodiment is the use of biguanides derivatives of formula I for the prevention and/or treatment of cancer, which comprises the administration of a therapeutically effective amount of one or more compounds of the formula I to a patient in need of such an administration, and where the patient also suffers from diabetes,more preferably from diabetes of type II (NIDDM).

Melanoma represents 5% of skin cancer but also 80% of death by skin cancer; incidence doubles every 10 years with 10000 new cases in France and more than 160000 worldwide.

Rising incidences of melanoma make it one of the rapidly growing cancers plaguing western populations. It is the 8^{th} leading cause of cancer deaths in the United States with an estimated 68,720 new cases diagnosed in 2009.

Current melanoma therapies such as surgery, radiation and chemotherapy are effective against early stage localized tumors. However, these therapies fail to treat and cure large malignant tumors which generally prove to be fatal. These therapies are also extremely damaging and toxic to the patient, suggesting a need for the development of new therapies.

In the new therapeutic approach, one the embodiment of this present invention relates to biguanides of formula I used for the treatment of melanomas without activation of AMPK pathway.

### BRIEF DESCRIPTION OF THE FIGURES

The FIG. 1 to FIG. 4 discloses this unexpected effect.

FIG. 1 discloses a study on viability cells of human melanomas cells (A375) in comparison to normal human melanocytes.

Doses of metformin used for this experiment are in a range between 0 to 20 mM at 72 h and 96 h.

FIG. 1′ discloses a study on viability cells of freshly human melanoma cells isolated from patients.

Doses of metformin used for this experiment are in a range between 0 to 10 mM at 72 h.

Metformin has a dose dependent effect on melanoma cells. By contrast, metformin has no effect on melanocytes (normal cells).

FIG. 1″ indicates that metformin induces a dose dependent cell death. Cell death was measured by FACS analysis of propidium iodide uptake.

FIG. 2 shows a dose dependent effect of metformin (dose 0 to 20 mM) on human G361 cells. AICAR [aminoimidazole carboxamide ribonucleotide] an AMPK agonist, is used as reference.

AICAR has no significative effect on the percentage of cell viability. Metformin shows a significant dose dependent decrease on the viability of G361 cells. FIG. 3:

This Western Blot shows that metformin effect on cell viability is independent on AMPK since AMPK disruption using si-RNA did not modify cell viability.

FIG. 4 shows the anti-melanoma activity of metformin in a mouse model of melanoma xenograft.A375 melanoma cells (2.5×10⁶) were injected subcutaneously in athymic nude mice and treated 5 days later by injection of vehicle or metformin (2 mg/mouse/day) over a period of 3 weeks.

Metformin decreases significantly the volume and the weight of melanoma after 23 days of treatment.

FIG. 4′ shows a representative TUNEL assay of mouse tumor sections as determination of cell death.

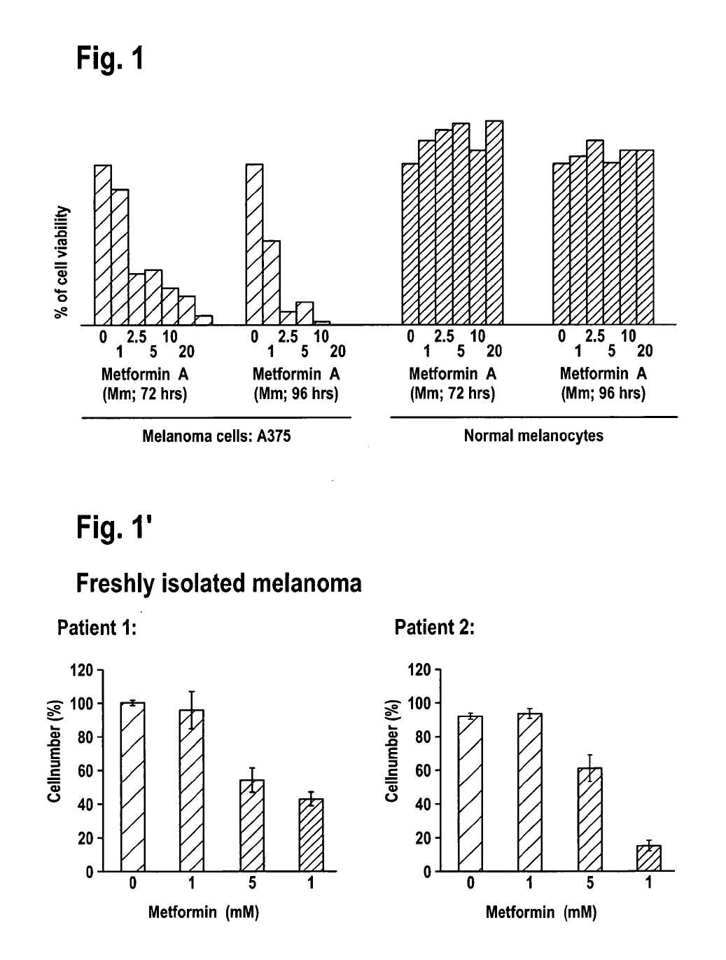

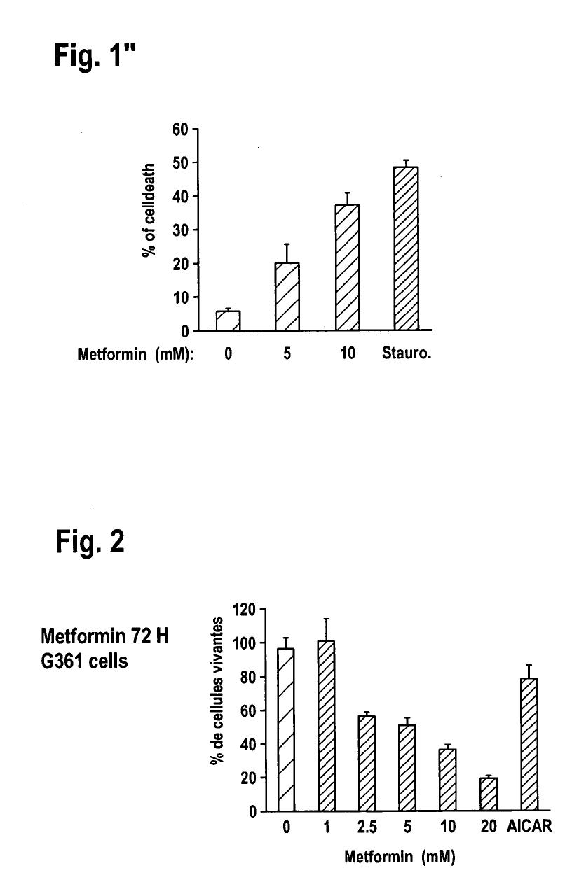

Experiment shows an increase in TUNEL-positive cells in the tumors of metformin-treated mice indicating that in vivo metformin induces death of melanoma cells.

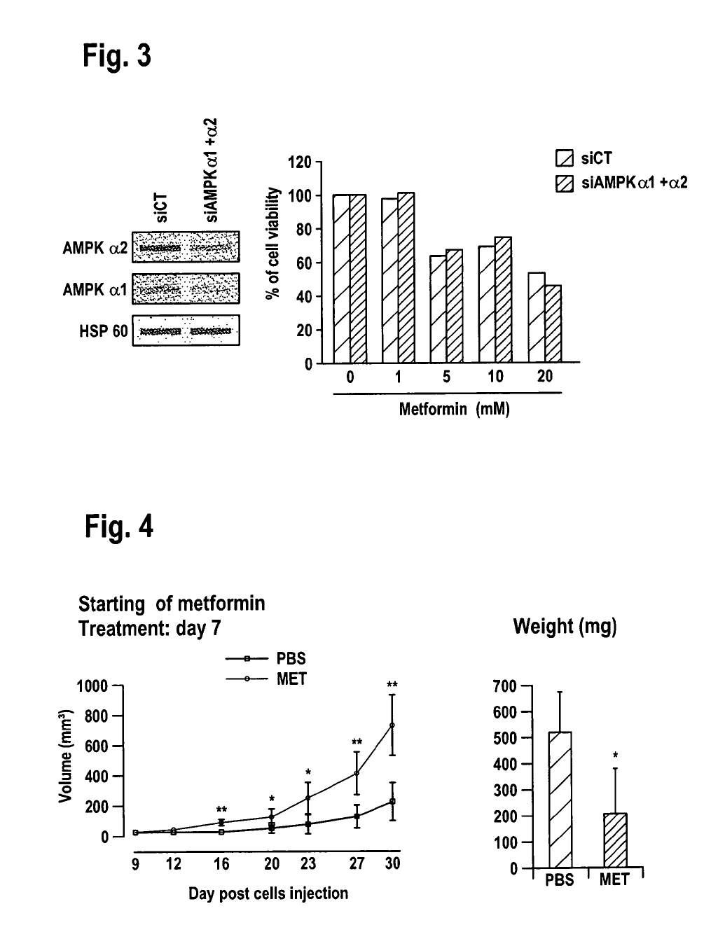

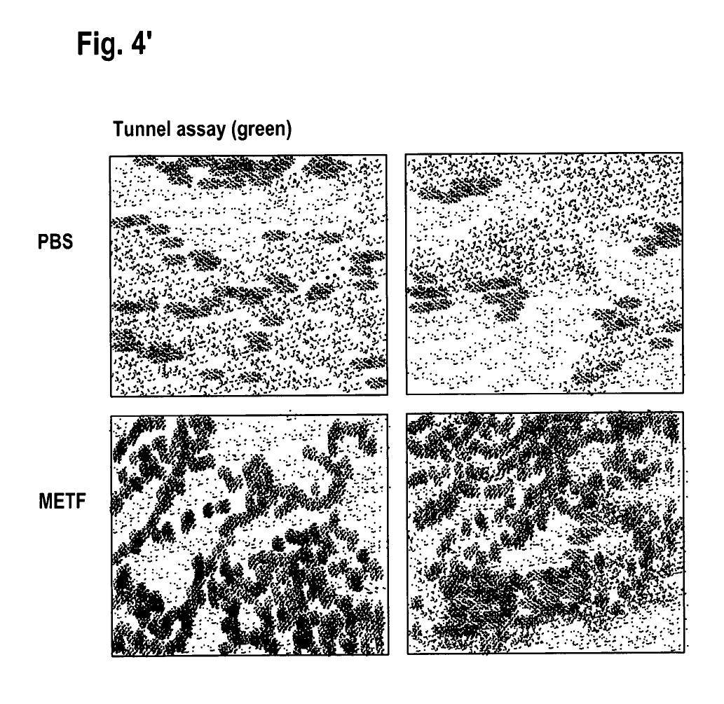

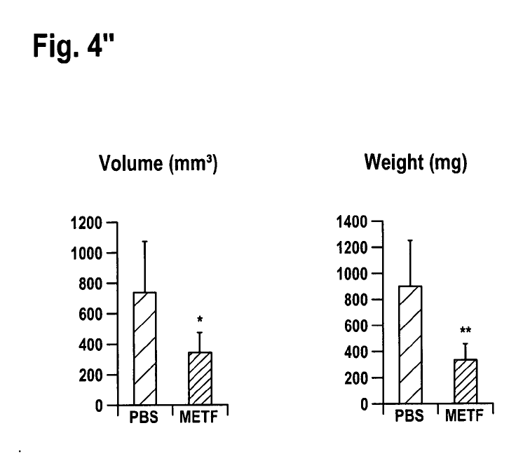

Finally, to circumvent partial immunity deficiency found in nude mice, we performed the same experiment in a syngenic model using allograft of B16 melanoma cells in C57B16 mice (FIG. 4″).

In this model also, metformin diminished the volume and weight of tumor of treated mice confirming the antineoplastic effect of this compound in vivo. The present invention therefore relates to compounds according to the invention as medicaments and/or medicament active ingredients in the treatment and/or prophylaxis of the said diseases and to the use of compounds according to the invention for the preparation of a pharmaceutical for the treatment and/or prophylaxis of the said diseases and also to a process for the treatment of the said diseases which comprises the administration of one or more compounds according to the invention to a patient in need of such an administration.

Surprisingly we have found that biguanides derivatives can be used for the treatment of cancer without activation of the known AMPK pathway (WO2005/023202) which has a key position in Glucose homeostasie.

These compounds are especially suitable for the prevention and treatment of cancer. It has been found that the compounds according to the invention and salts thereof have very valuable pharmacological properties while being well tolerated. The host or patient may belong to any mammal species, for example a primate species, particularly humans; rodents, including mice, rats and hamsters; rabbits; horses, cows, dogs, cats, etc. Animal models are of interest for experimental investigations, where they provide a model for the treatment of a human disease.

The invention relates to Compound of formula I

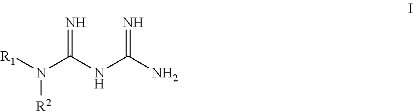

in which
R¹, R² each, independently of one another, denote H, A, Alk, (CH₂)ₙAr, (CH₂)ₙCyc or (CH₂)ₙHet,
R¹ and R² together also denote an alkylene chain having 2, 3, 4, 5 or 6 C atoms, in which one CH₂ group may be replaced by O, S, SO, SO₂, NH, NR⁸, NCOR⁸ or NCOOR⁸,
   and wherein the alkylene chain is unsubstituted or mono-, di- or trisubstituted by Hal, A, OR¹¹, N(R¹¹)₂, NO₂, CN, phenyl, ═O, CON(R¹¹)₂, NR¹¹COA, NR¹¹CON(R¹¹)₂, NR¹¹SO₂A, COR¹¹, SO₂N(R¹¹)₂, S(O)ₘA, —[C(R¹¹)₂]ₙ—COOR¹¹ and/or —O[C(R¹¹)₂]ₒ—COOR¹¹,
A denotes unbranched or branched alkyl having 1-10 C atoms, in which one, two or three CH and/or CH₂ groups may be replaced by O, S, SO, SO₂, NH, NR⁸ and/or by —CH═CH— groups and/or, in addition, 1-5 H atoms may be replaced by F, Cl, Br and/or R⁷,
Cyc cycloalkyl having 3-7 C atoms,
Alk denotes alkenyl or alkinyl having 2-6 C atoms,
R⁷ denotes COOR⁹, CONR⁹R¹⁰, NR⁹R¹⁰, NHCOR⁹, NHCOOR⁹ or OR⁹,
R⁸ denotes cycloalkyl having 3-7 C atoms, cycloalkylalkylene having 4-10 C atoms, Alk or
   unbranched or branched alkyl having 1-6 C atoms,
R⁹, R¹⁰ each, independently of one another, denote H or alkyl having 1-6 C atoms, in which 1-3 CH₂ groups may be replaced by O, S, SO, SO₂, NH, NMe or NEt and/or, in addition, 1-5 H atoms may be replaced by F and/or Cl,
Ar denotes phenyl, naphthyl or biphenyl, each of which is un-substituted or mono-, di- or trisubstituted by Hal, A, OR¹¹, N(R¹¹)₂, NO₂, CN, phenyl, CON(R¹¹)₂, NR¹¹COA, NR¹¹CON(R¹¹)₂, NR¹¹SO₂A, COR¹¹, SO₂N(R¹¹)₂, S(O)ₘA, —[C(R¹¹)₂]ₙ—COOR¹¹ and/or —O[C(R¹¹)₂]ₒ—COOR¹¹,
Het denotes a mono- or bicyclic saturated, unsaturated or aromatic heterocycle having 1 to 4 N, O and/or S atoms, which may be mono-, di- or trisubstituted by Hal, A, OR¹¹, N(R¹¹)₂, NO₂, CN, COOR¹¹, CON(R¹¹)₂, NR¹¹COA, NR¹¹SO₂A, COR¹¹, SO₂NR¹¹, S(O)ₘA, ═S, ═NR¹¹ and/or ═O (carbonyl oxygen),
R¹¹ denotes H or A,
Hal denotes F, Cl, Br or I,
m denotes 0, 1 or 2,
n denotes 0, 1, 2, 3 or 4,
o denotes 1, 2 or 3,
   with the exclusion of the compounds of formula I in which:

a-R¹═H, R²═H;

b-R¹═H, R²=phenethyl;

and pharmaceutically usable salts, solvates, tautomers and stereoisomers thereof, including mixtures thereof in all ratios,
for the use for the prevention and/or treatment of cancer.

The invention also relates to the hydrates and solvates of these compounds. Solvates of the compounds are taken to mean adductions of inert solvent molecules onto the compounds which form owing to their mutual attractive force. Solvates are, for example, mono- or dihydrates or alcoholates.

The expression “effective amount” means the amount of a medicament or pharmaceutical active ingredient which causes a biological or medical response which is sought or desired, for example, by a researcher or physician in a tissue, system, animal or human.

In addition, the expression “therapeutically effective amount” means an amount which, compared with a corresponding subject who has not received this amount, has the following consequence:

improved healing treatment, healing, prevention or elimination of a disease, a disease picture, a disease state, a complaint, a disorder or of side effects or also the reduction in the progress of a disease, a complaint or a disorder.

The term “therapeutically effective amount” also encompasses the amounts which are effective for increasing normal physiological function.

The invention also relates to mixtures of the compounds of the formula I according to the invention, for example mixtures of two diastereomers, for example in the ratio 1:1, 1:2, 1:3, 1:4, 1:5, 1:10, 1:100 or 1:1000.

These are particularly preferably mixtures of stereoisomeric compounds.

For all radicals which occur more than once, their meanings are independent of one another.

Above and below, the radicals and parameters R¹, R² have the meanings indicated for the formula I, unless expressly indicated otherwise.

A preferably denotes alkyl, is unbranched (linear) or branched, and has 1, 2, 3, 4, 5, 6, 7, 8, 9 or 10 C atoms. A particularly preferably denotes denotes methyl, ethyl, propyl, isopropyl, butyl, isobutyl, sec-butyl or tert-butyl, furthermore also pentyl, 1-, 2- or 3-methylbutyl, 1,1-, 1,2- or 2,2-dimethylpropyl, 1-ethylpropyl, hexyl, 1-, 2-, 3- or 4-methylpentyl, 1,1-, 1,2-, 1,3-, 2,2-, 2,3- or 3,3-dimethylbutyl, 1- or 2-ethylbutyl, 1-ethyl-1-methylpropyl, 1-ethyl-2-methylpropyl, 1,1,2- or 1,2,2-trimethylpropyl.

A very particularly preferably denotes alkyl having 1, 2, 3, 4, 5 or 6 C atoms, preferably ethyl, propyl, isopropyl, butyl, isobutyl, sec-butyl, tert-butyl, pentyl, hexyl, trifluoromethyl, pentafluoroethyl or 1,1,1-trifluoroethyl, furthermore also fluoromethyl, difluoromethyl or bromomethyl.

Cycloalkyl preferably denotes cyclopropyl, cyclobutyl, cyclopentyl, cyclohexyl or cycloheptyl.

Alk denotes alkenyl or alkinyl having 2-6 C atoms, such as, for example, vinyl or propenyl.

Cycloalkylalkylene denotes, for example, cyclohexylmethyl, cyclohexylethyl, cyclopentylmethyl or cyclopentylethyl.

Ar denotes, for example, phenyl, o-, m- or p-tolyl, o-, m- or p-ethylphenyl, o-, m- or p-propylphenyl, o-, m- or p-isopropylphenyl, o-, m- or p-tert-butylphenyl, o-, m- or p-hydroxyphenyl, o-, m- or p-nitrophenyl, o-, m- or p-aminophenyl, o-, m- or p-(N-methylamino)phenyl, o-, m- or p-(N-methylaminocarbonyl)phenyl, o-, m- or p-acetamidophenyl, o-, m- or p-methoxyphenyl, o-, m- or p-ethoxyphenyl, o-, m- or p-ethoxycarbonylphenyl, o-, m- or p-(N,N-dimethylamino)phenyl, o-, m- or p-(N,N-dimethylaminocarbonyl)phenyl, o-, m- or p-(N-ethylamino)phenyl, o-, m- or p-(N,N-diethylamino)phenyl, o-, m- or p-fluorophenyl, o-, m- or p-bromophenyl, o-, m- or p-chlorophenyl, o-, m- or p-(methylsulfonamido)phenyl, o-, m- or p-(methylsulfonyl)-phenyl, o-, m- or p-cyanophenyl, o-, m- or p-ureidophenyl, o-, m- or p-formylphenyl, o-, m- or p-acetylphenyl, o-, m- or p-aminosulfonylphenyl, o-, m- or p-carboxyphenyl, o-, m- or p-carboxymethylphenyl, o-, m- or p-carboxymethoxyphenyl, further preferably 2,3-, 2,4-, 2,5-, 2,6-, 3,4- or 3,5-difluorophenyl, 2,3-, 2,4-, 2,5-, 2,6-, 3,4- or 3,5-dichiorophenyl, 2,3-, 2,4-, 2,5-, 2,6-, 3,4- or 3,5-dibromophenyl, 2,4- or 2,5-dinitrophenyl, 2,5- or 3,4-dimethoxyphenyl, 3-nitro-4-chlorophenyl, 3-amino-4-chloro-, 2-amino-3-chloro-, 2-amino-4-chloro-, 2-amino-5-chloro- or 2-amino-6-chlorophenyl, 2-nitro-4-N,N-dimethylamino- or 3-nitro-4-N,N-dimethylaminophenyl, 2,3-diaminophenyl, 2,3,4-, 2,3,5-, 2,3,6-, 2,4,6- or 3,4,5-trichlorophenyl, 2,4,6-trimethoxyphenyl, 2-hydroxy-3,5-dichlorophenyl, p-iodophenyl, 3,6-dichloro-4-aminophenyl, 4-fluoro-3-chlorophenyl, 2-fluoro-4-bromophenyl, 2,5-difluoro-4-bromophenyl, 3-bromo-6-methoxyphenyl, 3-chloro-6-methoxyphenyl, 3-chloro-4-acetamidophenyl, 3-fluoro-4-methoxyphenyl, 3-amino-6-methylphenyl, 3-chloro-4-acetamidophenyl or 2,5-dimethyl-4-chlorophenyl.

Ar preferably denotes, for example, phenyl, which is unsubstituted or mono-, di- or trisubstituted by Hal, A, OR¹¹, N(R¹¹)₂, NO₂, CN, phenyl, and/or CON(R¹¹)₂.

Irrespective of further substitutions, Het denotes, for example, 2- or 3-furyl, 2- or 3-thienyl, 1-, 2- or 3-pyrrolyl, 1-, 2, 4- or 5-imidazolyl, 1-, 3-, 4- or 5-pyrazolyl, 2-, 4- or 5-oxazolyl, 3-, 4- or 5-isoxazolyl, 2-, 4- or 5-thiazolyl, 3-, 4- or 5-isothiazolyl, 2-, 3- or 4-pyridyl, 2-, 4-, 5- or 6-pyrimidinyl, furthermore preferably 1,2,3-triazol-1-, -4- or -5-yl, 1,2,4-triazol-1-, -3- or 5-yl, 1- or 5-tetrazolyl, 1,2,3-oxadiazol-4- or -5-yl, 1,2,4-oxadiazol-3- or -5-yl, 1,3,4-thiadiazol-2- or -5-yl, 1,2,4-thiadiazol-3- or -5-yl, 1,2,3-thiadiazol-4- or -5-yl, 3- or 4-pyridazinyl, pyrazinyl, 1-, 2-, 3-, 4-, 5-, 6- or 7-indolyl, 4- or 5-isoindolyl, 1-, 2-, 4- or 5-benzimidazolyl, 1-, 2-, 3-, 4-, 5-, 6- or 7-indazolyl, 1-, 3-, 4-, 5-, 6- or 7-benzopyrazolyl, 2-, 4-, 5-, 6- or 7-benzoxazolyl, 3-, 4-, 5-, 6- or 7- benzisoxazolyl, 2-, 4-, 5-, 6- or 7-benzothiazolyl, 2-, 4-, 5-, 6- or 7-benzisothiazolyl, 4-, 5-, 6- or 7-benz-2,1,3-oxadiazolyl, 2-, 3-, 4-, 5-, 6-, 7- or 8-quinolyl, 1-, 3-, 4-, 5-, 6-, 7- or 8-isoquinolyl, 3-, 4-, 5-, 6-, 7- or 8-cinnolinyl, 2-, 4-, 5-, 6-, 7- or 8-quinazolinyl, 5- or 6-quinoxalinyl, 2-, 3-, 5-, 6-, 7- or 8-2H-benzo-1,4-oxazinyl, further preferably 1,3-benzodioxol-5-yl, 1,4-benzodioxan-6-yl, 2,1,3-benzothiadiazol-4- or -5-yl or 2,1,3-benzoxadiazol-5-yl.

The heterocyclic radicals may also be partially or fully hydrogenated. Het can thus also denote, for example, 2,3-dihydro-2-, -3-, -4- or -5-furyl, 2,5-dihydro-2-, -3-, -4- or 5-furyl, tetrahydro-2- or -3-furyl, 1,3-dioxolan-4-yl, tetrahydro-2- or -3-thienyl, 2,3-dihydro-1-, -2-, -3-, -4- or -5-pyrrolyl, 2,5-dihydro-1-, -2-, -3-, -4- or -5-pyrrolyl, 1-, 2- or 3-pyrrolidinyl, tetrahydro-1-, -2- or -4-imidazolyl, 2,3-dihydro-1-, -2-, -3-, -4- or -5-pyrazolyl, tetrahydro-1-, -3- or -4-pyrazolyl, 1,4-dihydro-1-, -2-, -3- or -4-pyridyl, 1,2,3,4-tetrahydro-1-, -2-, -3-, -4-, -5- or -6-pyridyl, 1-, 2-, 3- or 4-piperidinyl, 2-, 3- or 4-morpholinyl, tetrahydro-2-, -3- or -4-pyranyl, 1,4-dioxanyl, 1,3-dioxan-2-, -4- or -5-yl, hexahydro-1-, -3- or -4-pyridazinyl, hexahydro-1-, -2-, -4- or -5-pyrimidinyl, 1-, 2- or 3-piperazinyl, 1,2,3,4-tetrahydro-1-, -2-, -3-, -4-, -5-, -6-, -7- or -8-quinolyl, 1,2,3,4-tetrahydro-1-,-2-,-3-, -4-, -5-, -6-, -7- or -8-isoquinolyl, 2-, 3-, 5-, 6-, 7- or 8-3,4-dihydro-2H-benzo-1,4-oxazinyl, further preferably 2,3-methylenedioxyphenyl, 3,4-methylenedioxyphenyl, 2,3-ethylenedioxyphenyl, 3,4-ethylenedioxyphenyl, 3,4-(difluoromethylenedioxy)phenyl, 2,3-dihydrobenzofuran-5- or 6-yl, 2,3-(2-oxomethylenedioxy)phenyl or also 3,4-dihydro-2H-1,5-benzodioxepin-6- or -7-yl, furthermore preferably 2,3-dihydrobenzofuranyl or 2,3-dihydro-2-oxofuranyl.

Het preferably denotes a monocyclic saturated, unsaturated or aromatic heterocycle having 1 to 2 N, O and/or S atoms, which may be mono-, di- or trisubstituted by Hal, A, and/or ═O (carbonyl oxygen).

In a further embodiment, Het very particularly preferably denotes pyrrolidinyl, piperidinyl, morpholinyl or piperazinyl, where the radicals may also be monosubstituted by A.

In a further embodiment, Het particularly preferably denotes furyl, thienyl, pyrrolyl, imidazolyl, pyridyl, pyrimidinyl, pyrazolyl, thiazolyl, indolyl, pyrrolidinyl, piperidinyl, morpholinyl or piperazinyl, each of which is unsubstituted or mono-, di- or trisubstituted by A, Hal, OH and/or OA.

R¹, R² preferably denote, each, independently of one another, H, A, Alk, (CH₂)ₙAr, (CH₂)ₙCyc or (CH₂)ₙHet; and R¹ and R²together also denote an alkylene chain having 2, 3, 4, 5 or 6 C atoms, in which one CH₂ group may be replaced by O, NH or NR⁸.

R¹, R² preferably also denote, each, independently of one another, alkyl having 1-4 C atoms.R¹, R² particularly preferably denote methyl.

R⁷ preferably denotes COOH, COOCH₃, COOC₂H₅, CONH₂, CON(CH₃)₂, NH₂, N(CH₃)₂, NHCOCH₃, OH or OCH₃.

R⁸ preferably denotes alkyl having 1, 2, 3 or 4 C atoms.

R⁹ preferably denotes H or alkyl having 1, 2, 3 or 4 C atoms.

R¹⁰ preferably denotes H or alkyl having 1, 2, 3 or 4 C atoms.

R¹¹ preferably denotes H or alkyl having 1, 2, 3 or 4 C atoms.

Accordingly, the invention relates, in particular, to the compounds of the formula I in which at least one of the said radicals has one of the preferred meanings indicated above. Some preferred groups of compounds may be expressed by the following sub-formulae la to If, which conform to the formula I and in which the radicals not designated in greater detail have the meaning indicated for the formula I, but in which
in Ia R¹, R² each, independently of one another, denote H, A, Alk, (CH₂)ₙAr, (CH₂)ₙCyc or (CH₂)ₙ, Het,
   R¹ and R² together also denote an alkylene chain having 2, 3, 4, 5 or 6 C atoms, in which one CH₂ group may be replaced by O, NH or NR⁸;
in Ib A denotes unbranched or branched alkyl having 1-6 C atoms, in which 1-5 H atoms may be replaced by F, Cl and/or Br;
in Ic Ar denotes phenyl, which is unsubstituted or mono-, di- or trisubstituted by Hal, A, OR¹¹, N(R¹¹)₂, NO₂, CN, phenyl, and/or CON(R¹¹)₂;
in Id Het denotes a monocyclic saturated, unsaturated or aromatic heterocycle having 1 to 2 N, O and/or S atoms, which may be mono-, di- or trisubstituted by Hal, A, and/or ═O (carbonyl oxygen);
in Ie R¹, R² each, independently of one another, denote alkyl having 1-4 C atoms;
in If R¹, R² denote methyl;
   with the exclusion of the compounds of formula I in which:

a-R¹═H, R²═H;

b-=H, R²=phenethyl;

and pharmaceutically usable salts, solvates, tautomers and stereoisomers thereof, including mixtures thereof in all ratios,
for the use for the prevention and/or treatment of cancer.

The invention also relates to a compound of formula I, in which

R¹, R² denote methyl, and pharmaceutically usable salts, solvates, tautomers and stereoisomers thereof, including mixtures thereof in all ratios, for the use for the prevention and/or treatment of melanoma.

The invention also relates to a compound of formula I, for the use for the prevention and/or treatment of cancer, which comprises the administration of one or more compounds of the formula I to a patient in need of such an administration, and where the patient also suffers from a disease selected from diabetes, dyslipidemia, hypertension, retinopathy, nephropathy, neuropathy, obesity connected with the Metabolic Syndrome.

Preferably, the invention also relates to a compound of formula I, for the use for the prevention and/or treatment of cancer, where the patient also suffers from diabetes. More preferably, the invention also relates to a compound of formula I, in which R¹, R² denote methyl, and pharmaceutically usable salts, solvates, tautomers and stereoisomers thereof, including mixtures thereof in all ratios,

for the use for the prevention and/or treatment of cancer, which comprises the administration of a therapeutically effective amount of one or more compounds of the formula I to a patient in need of such an administration, and where the patient also suffers from diabetes.

The invention also relates to a compound of formula I, and pharmaceutically usable salts, solvates, tautomers and stereoisomers thereof, including mixtures thereof in all ratios, for the use for the prevention and/or treatment of cancer, and at least one further medicament active ingredient.

The invention also relates to a compound of formula I, and pharmaceutically usable salts, solvates, tautomers and stereoisomers thereof, including mixtures thereof in all ratios,

for the use for the prevention and/or treatment of cancer, wherein a further medicament active ingredient is an anticancer agent.

The invention also relates to a compound of formula I, in which R¹, R² denote methyl, and pharmaceutically usable salts, solvates, tautomers and stereoisomers thereof, including mixtures thereof in all ratios,

for the use for the prevention and/or treatment of melanoma, wherein a further medicament active ingredient is an anticancer agent.

Most preferably metformin is used as hydrochloride salt for the treatment of cancer. The biguanides compounds of the formula (I) can be prepared by reaction of an amine R¹R²NH on 2-cyanoguanidine according to the literature [FR2322860,1975; J. Am. Chem. Soc.,(1959), 81,3728; Farmaco, Edizione Scientifico, (1964),19, 342; Indian Journal of Chemistry, section B, (1984), 23B, 789; Helvetica Chemica Acta, (1988), 71, 77; Zhurnal Organischeskoi Khimii, (1988), 24,1100]

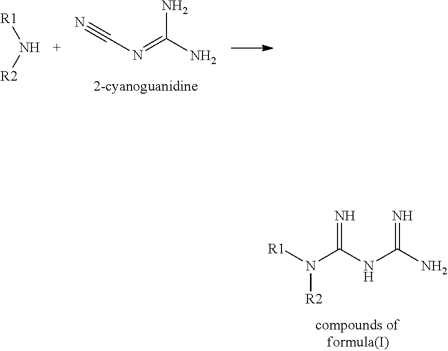

The compound according to formula (I) can be used for the production of medicaments comprising at least one compound of the formula (I) and/or pharmaceutically usable salts, tautomers and stereoisomers thereof, including mixtures thereof in all ratios, and optionally excipients and/or adjuvants, for the preparation of a medicament for the treatment of cancer.

The disclosed compounds of the formula I can be administered in combination with other known therapeutic agents, including anticancer agents. As used here, the term “anticancer agent” relates to any agent which is administered to a patient with cancer for the purposes of treating the cancer.

The anti-cancer treatment defined herein may be applied as a sole therapy or may involve, in addition to the compound of the invention, conventional surgery or radiotherapy or chemotherapy. Such chemotherapy may include one or more of the following categories of anti-tumour agents:

(i) antiproliferative/antineoplastic/DNA-damaging agents and combinations thereof, as used in medical oncology, such as alkylating agents (for example cis-platin, carboplatin, cyclophosphamide, nitrogen mustard, melphalan, chloroambucil, busulphan and nitrosoureas); antimetabolites (for example antifolates such as fluoropyrimidines like 5-fluorouracil and tegafur, raltitrexed, methotrexate, cytosine arabinoside, hydroxyurea and gemcitabine); antitumour antibiotics (for example anthracyclines, like adriamycin, bleomycin, doxorubicin, daunomycin, epirubicin, idarubicin, mitomycin-C, dactinomycin and mithramycin); antimitotic agents (for example vinca alkaloids, like vincristine, vinblastine, vindesine and vinorelbine, and taxoids, like taxol and taxotere) ; topoisomerase inhibitors (for example epipodophyllotoxins, like etoposide and teniposide, amsacrine, topotecan, irinotecan and camptothecin) and cell-differentiating agents (for example all-trans-retinoic acid, 13-cis-retinoic acid and fenretinide);

(ii) cytostatic agents, such as antioestrogens (for example tamoxifen, toremifene, raloxifene, droloxifene and iodoxyfene), oestrogen receptor downregulators (for example fulvestrant), antiandrogens (for example bicalutamide, flutamide, nilutamide and cyproterone acetate), LHRH antagonists or LHRH agonists (for example goserelin, leuprorelin and buserelin), progesterones (for example megestrol acetate), aromatase inhibitors (for example as anastrozole, letrozole, vorazole and exemestane) and inhibitors of 5α-reductase, such as finasteride;

(iii) agents which inhibit cancer cell invasion (for example metalloproteinase inhibitors, like marimastat, and inhibitors of urokinase plasminogen activator receptor function);

(iv) vessel-damaging agents, such as combretastatin A4 and compounds disclosed in international patent applications WO 99/02166, WO 00/40529, WO 00/41669, WO 01/92224, WO 02/04434 and WO 02/08213;

(v) antisense therapies, for example those which are directed to the targets listed above, such as ISIS 2503, an anti-Ras antisense;

(vi) gene therapy approaches, including, for example, approaches for replacement of aberrant genes, such as aberrant p53 or aberrant BRCA1 or BRCA2, GDEPT (gene-directed enzyme pro-drug therapy) approaches, such as those using cytosine deaminase, thymidine kinase or a bacterial nitroreductase enzyme, and approaches for increasing patient tolerance to chemotherapy or radiotherapy, such as multi-drug resistance gene therapy; and

(vii) immunotherapy approaches, including, for example, ex-vivo and in-vivo approaches for increasing the immunogenicity of patient tumour cells, such as transfection with cytokines, such as interleukin 2, interleukin 4 or granulocyte-macrophage colony stimulating factor, approaches for decreasing T-cell anergy, approaches using transfected immune cells, such as cytokine-transfected dendritic cells, approaches using cytokine-transfected tumour cell lines, and approaches using anti-idiotypic antibodies.

The medicaments from Table 1 below are preferably, but not exclusively, combined with the compounds of the formula I.

TABLE 1

| | | |
|---|---|---|
| Alkylating agents | Cyclophosphamide | Lomustine |
| | Busulfan | Procarbazine |
| | Ifosfamide | Altretamine |
| | Melphalan | Estramustine phosphate |
| | Hexamethylmelamine | Mechloroethamine |
| | Thiotepa | Streptozocin |
| | chloroambucil | Temozolomide |
| | Dacarbazine | Semustine |
| | Carmustine | |
| Platinum agents | Cisplatin | Carboplatin |
| | Oxaliplatin | ZD-0473 (AnorMED) |
| | Spiroplatin | Lobaplatin (Aetema) |
| | Carboxyphthalatoplatinum | Satraplatin (Johnson |
| | Tetraplatin | Matthey) |
| | Ormiplatin | BBR-3464 |
| | Iproplatin | (Hoffrnann-La Roche) |
| | | SM-11355 (Sumitomo) |
| | | AP-5280 (Access) |
| Antimetabolites | Azacytidine | Tomudex |
| | Gemcitabine | Trimetrexate |
| | Capecitabine | Deoxycoformycin |
| | 5-fluorouracil | Fludarabine |
| | Floxuridine | Pentostatin |
| | 2-chlorodesoxyadenosine | Raltitrexed |
| | 6-Mercaptopurine | Hydroxyurea |
| | 6-Thioguanine | Decitabine (SuperGen) |
| | Cytarabine | Clofarabine (Bioenvision) |
| | 2-fluorodesoxycytidine | Irofulven (MGI Pharrna) |
| | Methotrexate | DMDC (Hoffmann-La |
| | Idatrexate | Roche) |
| | | Ethynylcytidine (Taiho ) |
| Topoisomerase | Amsacrine | Rubitecan (SuperGen) |
| inhibitors | Epirubicin | Exatecan mesylate |
| | Etoposide | (Daiichi) |
| | Teniposide or mitoxantrone | Quinamed (ChemGenex) |
| | Irinotecan (CPT-11) | Gimatecan (Sigma-Tau) |
| | 7-ethyl-10-hydroxycamptothecin | Diflomotecan (Beaufour- |
| | Topotecan | Ipsen) |
| | Dexrazoxanet (TopoTarget) | TAS-103 (Taiho) |
| | Pixantrone (Novuspharrna) | Elsamitrucin (Spectrum) |
| | Rebeccamycin analogue (Exelixis) | J-107088 (Merck & Co) |
| | BBR-3576 (Novuspharrna) | BNP-1350 (BioNumerik) |
| | | CKD-602 (Chong Kun |
| | | Dang) |
| | | KW-2170 (Kyowa Hakko) |
| Antitumour antibiotics | Dactinomycin (Actinomycin D) | Amonafide |
| | Doxorubicin (Adriamycin) | Azonafide |
| | Deoxyrubicin | Anthrapyrazole |
| | Valrubicin | Oxantrazole |
| | Daunorubicin (Daunomycin) | Losoxantrone |
| | Epirubicin | Bleomycin sulfate |
| | Therarubicin | (Blenoxan) |
| | Idarubicin | Bleomycinic acid |
| | Rubidazon | Bleomycin A |
| | Plicamycinp | Bleomycin B |
| | Porfiromycin | Mitomycin C |
| | Cyanomorpholinodoxorubicin | MEN-10755 (Menarini) |
| | Mitoxantron (Novantron) | GPX-100 (Gem |
| | | Pharmaceuticals) |
| Antimitotic agents | Paclitaxel | SB 408075 |
| | Docetaxel | (GlaxoSmithKline) |
| | Colchicine | E7010 (Abbott) |
| | Vinblastine | PG-TXL (Cell |
| | Vincristine | Therapeutics) |
| | Vinorelbine | IDN 5109 (Bayer) |
| | Vindesine | A 105972 (Abbott) |
| | Dolastatin 10 (NCI) | A 204197 (Abbott) |
| | Rhizoxin (Fujisawa) | LU 223651 (BASF) |
| | Mivobulin (Warner-Lambert) | D 24851 (ASTA Medica) |
| | Cemadotin (BASF) | ER-86526 (Eisai) |
| | RPR 109881A (Aventis) | Combretastatin A4 |
| | TXD 258 (Aventis) | (BMS) |
| | Epothilone B (Novartis) | Isohomohalichondrin-B |
| | T 900607 (Tularik) | (PharmaMar) |
| | T 138067 (Tularik) | ZD 6126 (AstraZeneca) |
| | Cryptophycin 52 (Eli Lilly) | PEG-Paclitaxel (Enzon) |
| | Vinflunine (Fabre) | AZ10992 (Asahi) |
| | Auristatin PE (Teikoku Hormone) | IDN-5109 (Indena) |
| | BMS 247550 (BMS) | AVLB (Prescient |
| | BMS 184476 (BMS) | NeuroPharma) |
| | BMS 188797 (BMS) | Azaepothilon B (BMS) |
| | Taxoprexin (Protarga) | BNP-7787 (BioNumerik) |
| | | CA-4-prodrug |
| | | (OXiGENE) |
| | | Dolastatin-10 (NrH) |
| | | CA-4 (OXiGENE) |
| Aromatase inhibitors | Aminoglutethimide | Exemestan |
| | Letrozole | Atamestan |
| | Anastrazole | (BioMedicines) |
| | Formestan | YM-511 (Yamanouchi) |
| Thymidylate synthase | Pemetrexed (Eli Lilly) | Nolatrexed (Eximias) |
| inhibitors | ZD-9331 (BTG) | CoFactor ™ (BioKeys) |
| DNA antagonists | Trabectedin (PharmaMar) | Mafosfamide (Baxter |
| | Glufosfamide (Baxter International) | International) |
| | Albumin + 32P (Isotope Solutions) | Apaziquone (Spectrum |
| | Thymectacin (NewBiotics) | Pharmaceuticals) |
| | Edotreotid (Novartis) | O6-benzylguanine |
| | | (Paligent) |
| Farnesyl transferase | Arglabin (NuOncology Labs) | Tipifarnib (Johnson & |
| inhibitors | Ionafarnib (Schering-Plough) | Johnson) |
| | BAY-43-9006 (Bayer) | Perillyl alcohol (DOR |
| | | BioPharma) |
| Pump inhibitors | CBT-1 (CBA Pharma) | Zosuquidar |
| | Tariquidar (Xenova) | trihydrochloride (Eli Lilly) |
| | MS-209 (Schering AG) | Biricodar dicitrate |
| | | (Vertex) |
| Histone acetyl | Tacedinaline (Pfizer) | Pivaloyloxymethyl |
| transferase inhibitors | SAHA (Aton Pharma) | butyrate (Titan) |
| | MS-275 (Schering AG) | Depsipeptide (Fujisawa) |
| Metalloproteinase | Neovastat (Aeterna Laboratories) | CMT-3 (CollaGenex) |
| inhibitors | Marimastat (British Biotech) | BMS-275291 (Celltech) |
| Ribonucleoside | Gallium maltolate (Titan) | Tezacitabine (Aventis) |
| reductase inhibitors | Triapin (Vion) | Didox (Molecules for |
| | | Health) |
| TNF-alpha | Virulizin (Lorus Therapeutics) | Revimid (Celgene) |
| agonists/ | CDC-394 (Celgene) | |
| antagonists | | |
| Endothelin-A receptor | Atrasentan (Abbot) | YM-598 (Yamanouchi) |
| antagonists | ZD-4054 (AstraZeneca) | |
| Retinoic acid receptor | Fenretinide (Johnson & Johnson) | Alitretinoin (Ligand) |
| agonists | LGD-1550 (Ligand) | |
| Immunomodulators | Interferon | Dexosome therapy |
| | Oncophage (Antigenics) | (Anosys) |
| | GMK (Progenics) | Pentrix (Australian |
| | Adenocarcinoma vaccine (Biomira) | Cancer |
| | CTP-37 (AVI BioPharma) | Technology) |
| | JRX-2 (Immuno-Rx) | JSF-154 (Tragen) |
| | PEP-005 (Peplin Biotech) | Cancer vaccine |
| | Synchrovax vaccines (CTL Immuno) | (Intercell) |
| | Melanoma vaccine (CTL Immuno) | Norelin (Biostar) |
| | p21-RAS vaccine (GemVax) | BLP-25 (Biomira) |
| | | MGV (Progenics) |
| | | !3-Alethin (Dovetail) |
| | | CLL-Thera (Vasogen) |
| Hormonal and | Oestrogens | Prednisone |
| antihormonal agents | Conjugated oestrogens | Methylprednisolone |
| | Ethynyloestradiol | Prednisolone |
| | chlorotrianisene | Aminoglutethimide |
| | Idenestrol | Leuprolide |
| | Hydroxyprogesterone caproate | Goserelin |
| | Medroxyprogesterone | Leuporelin |
| | Testosterone | Bicalutamide |
| | Testosterone propionate | Flutamide |
| | Fluoxymesterone | Octreotide |
| | Methyltestosterone | Nilutamide |
| | Diethylstilbestrol | Mitotan |
| | Megestrol | P-04 (Novogen) |
| | Tamoxifen | 2-Methoxyoestradiol |
| | Toremofin | (EntreMed) |
| | Dexamethasone | Arzoxifen (Eli Lilly) |
| Photodynamic agents | Talaporfin (Light Sciences) | Pd-Bacteriopheophorbid |
| | Theralux (Theratechnologies) | (Yeda) |
| | Motexafin-Gadolinium | Lutetium-Texaphyrin |
| | (Pharmacyclics) | (Pharmacyclics) |
| | | Hypericin |
| Various agents | SR-27897 (CCK-A inhibitor, Sanofi- | BCX-1777 (PNP |
| | Synthelabo) | inhibitor, BioCryst) |
| | Tocladesine (cyclic AMP agonist, | Ranpirnase |
| | Ribapharm) | (ribonuclease stimulant, |
| | Alvocidib (CDK inhibitor, Aventis) | Alfacell) |
| | CV-247 (COX-2 inhibitor, Ivy | Galarubicin (RNA |
| | Medical) | synthesis inhibitor, Dong- |
| | P54 (COX-2 inhibitor, Phytopharm) | A) |
| | CapCell ™ (CYP450 stimulant, | Tirapazamine (reducing |
| | Bavarian Nordic) | agent, SRI International) |
| | GCS-IOO (gal3 antagonist, | N-Acetylcysteine |
| | GlycoGenesys) | (reducing agent, |
| | G17DT immunogen (gastrin inhibitor, | Zambon) |
| | Aphton) | R-Flurbiprofen (NF-kap- |
| | Efaproxiral (oxygenator, Allos | paB inhibitor, Encore) |
| | Therapeutics) | 3CPA (NF-kappaB |
| | PI-88 (heparanase inhibitor, Progen) | inhibitor, Active Biotech) |
| | Tesmilifen (histamine antagonist, YM | Seocalcitol (vitamin D |
| | BioSciences) | receptor agonist, Leo) |
| | Histamine (histamine H2 receptor | 131-1-TM-601 (DNA |
| | agonist, Maxim) | antagonist, |
| | Tiazofurin (IMPDH inhibitor, | TransMolecular) |
| | Ribapharm) | Eflornithin (ODC |
| | Cilengitide (integrin antagonist, | inhibitor, ILEX Oncology) |
| | Merck KGaA) | Minodronic acid |
| | SR-31747 (IL-1 antagonist, Sanofi- | (osteoclast inhibitor, |
| | Synthelabo) | Yamanouchi) |
| | CCI-779 (mTOR kinase inhibitor, | Indisulam (p53 stimulant, |
| | Wyeth) | Eisai) |
| | Exisulind (PDE-V inhibitor, Cell | Aplidin (PPT inhibitor, |
| | Pathways) | PharmaMar) |
| | CP-461 (PDE-V inhibitor, Cell | Rituximab (CD20 |
| | Pathways) | antibody, Genentech) |
| | AG-2037 (GART inhibitor, Pfizer) | Gemtuzumab (CD33 |
| | WX-UK1 (plasminogen activator | antibody, Wyeth Ayerst) |
| | inhibitor, Wilex) | PG2 (haematopoiesis |
| | PBI-1402 (PMN stimulant, ProMetic | promoter, |
| | LifeSciences) | Pharmagenesis) |
| | Bortezomib (proteasome inhibitor, | Immunol ™ (triclosan |
| | Millennium) | mouthwash, Endo) |
| | SRL-172 (T-cell stimulant, SR | Triacetyluridine (uridine |
| | Pharma) | prodrug, Wellstat) |
| | TLK-286 (glutathione-S transferase | SN-4071 (sarcoma |
| | inhibitor, Telik) | agent, Signature |
| | PT-100 (growth factor agonist, Point | BioScience) |
| | Therapeutics) | TransMID-107 ™ |
| | Midostaurin (PKC inhibitor, Novartis) | (immunotoxin, KS |
| | Bryostatin-1 (PKC stimulant, GPC | Biomedix) |
| | Biotech) | PCK-3145 (apoptosis |
| | CDA-II (apoptosis promoter, Everlife) | promoter, Procyon) |
| | SDX-101 (apoptosis promoter, | Doranidazole (apoptosis |
| | Salmedix) | promoter, Pola) |
| | Ceflatonin (apoptosis promoter, | CHS-828 (cytotoxic |
| | ChemGenex) | agent, Leo) |
| | | Trans-retinic acid |
| | | (differentiator, NIH) |
| | | MX6 (apoptosis |

A combined treatment of this type can be achieved with the aid of simultaneous, consecutive or separate dispensing of the individual components of the treatment. Combination products of this type employ the compounds according to the invention.

The composition of one compound of the formula (I) and at least one further medicament active ingredient can be combined in a Set (kit) consisting of separate packs of
(a) an effective amount of a compound of the formula I, and pharmaceutically usable solvates, salts and stereoisomers thereof, including mixtures thereof in all ratios, and
(b) an effective amount of a further medicament active ingredient.

In the meaning of the present invention, the compound of formula (I) is defined to include pharmaceutically usable derivatives comprising solvates, salts, tautomers, enantiomers, racemates and stereoisomers thereof, including mixtures thereof in all ratios. Preference is given to solvates and/or physiologically acceptable salts, more preferably physiologically acceptable salts, most preferably physiologically acceptable acid-addition salts.

The term “solvates” of the biguanide derivatives is taken to mean adductions of inert solvent molecules onto the compounds, which are formed owing to their mutual attractive force. Solvates are, for example, mono- or dihydrates or alkoxides.

The term “prodrug” is taken to mean compounds according to the invention which have been modified by means of, for example, alkyl or acyl groups, sugars or oligopeptides and which are rapidly cleaved in the organism to form the effective compounds according to the invention. These also include biodegradable polymer derivatives of the compounds according to the invention, as described, for example, in Int. J. Pharm. 115: 61-67 (1995). The compound of the invention can be obtained by liberating it from their functional derivatives by solvolysis, in particular hydrolysis, or by hydrogenolysis. It is likewise possible for the compounds of the invention to be in the form of any desired prodrugs, such as esters, carbonates, carbamates, ureas, amides or phosphates, in which cases the actually biologically active form is released only through metabolism. Any compound that can be converted in-vivo to provide the bioactive agent (i.e. compounds of the invention) is a prodrug within the scope and spirit of the invention. Various forms of prodrugs are well known in the art and are described (e.g. Wermuth et al. (1996) The Practice of Medicinal Chemistry, Chapter 31: 671-696, Academic Press; Bundgaard, H. (1985) Design of Prodrugs, Elsevier; Bundgaard, H. (1991) A Textbook of Drug Design and Development, Chapter 5: 131-191, Harwood Academic Publishers). It is further known that chemical substances are converted in the body into metabolites which may where appropriate likewise elicit the desired biological effect—in some circumstances even in more pronounced form. Any biologically active compound that was converted in-vivo by metabolism from any of the compounds of the invention is a metabolite within the scope and spirit of the invention.

The compounds of the invention may be present in the form of their double bond isomers as pure E or Z isomers, or in the form of mixtures of these double bond isomers. Where possible, the compounds of the invention may be in the form of the tautomers, such as keto-enol tautomers.

Formula (I) also encompasses the optically active forms (stereoisomers), such as the enantiomers. All stereoisomers of the compounds of the invention are contemplated, either in a mixture or in pure or substantially pure form. The compounds of the invention can have asymmetric centers at any of the carbon atoms. Consequently, they can exist in the form of their racemates, in the form of the pure enantiomers and/or diastereomers or in the form of mixtures of these enantiomers and/or diastereomers. The mixtures may have any desired mixing ratio of the stereoisomers. For example, the compounds of the invention which have one or more centers of chirality and which occur as racemates or as diastereomer mixtures can be fractionated by methods known per se into their optical pure isomers, i.e. enantiomers or diastereomers. The separation of the compounds of the invention can take place by column separation on chiral or non-chiral phases or by re-crystallization from an optionally optically active solvent or with use of an optically active acid or base or by derivatization with an optically active reagent such as, for example, an optically active alcohol, and subsequent elimination of the radical.

The invention also relates to the use of mixtures of the compounds according to the invention, for example mixtures of two diastereomers, for example in the ratio 1:1, 1:2, 1:3, 1:4, 1:5; 1:10, 1:100 or 1:1000. These are particularly preferably mixtures of stereoisomeric compounds. The composition may also comprise mixtures of the compound and at least a singe derivative, or mixtures of derivatives, respectively, which may comprise solvates and/or salts, for instance.

The nomenclature as used herein for defining compounds of formula(I) is biguanide compound referring to the name used for metformine (dimethylbiguanide, hydrochloride salt). UIPAC organization can define the biguanide compound with this following name:

Imidodicarbonimidic diamide with for exemple of metformin compound: N,N-dimethylimidodicarbonimidic diamide (dimethylbiguanide).

The biguanide derivatives according to formula (I) and the starting materials for its preparation, respectively, are produced by methods known per se, as described in the literature (for example in standard works, such as Houben-Weyl, Methoden der organischen Chemie [Methods of Organic Chemistry], Georg-Thieme-Verlag, Stuttgart), i.e. under reaction conditions that are known and suitable for said reactions. Use can also be made of variants that are known per se, but are not mentioned in greater detail herein. If desired, the starting materials can also be formed in-situ by leaving them in the un-isolated status in the crude reaction mixture, but immediately converting them further into the compound according to the invention. On the other hand, it is possible to carry out the reaction stepwise.

The said compounds formula (I) according to the invention can be used in their final non-salt form. On the other hand, the present invention also encompasses the use of these compounds in the form of their pharmaceutically acceptable salts, which can be derived from various organic and inorganic acids and bases by procedures known in the art. Pharmaceutically acceptable salt forms of the compounds according to the invention are for the most part prepared by conventional methods.

The reaction is preferably carried out in the presence of an organic or inorganic acid. Thus, it is possible to use inorganic acids, for example sulfuric acid, nitric acid, hydrohalic acids, such as hydrochloric acid or hydrobromic acid, phosphoric acids, such as orthophosphoric acid, sulfamic acid, furthermore organic acids, in particular aliphatic, alicyclic, araliphatic, aromatic or heterocyclic mono- or polybasic carboxylic, sulfonic or sulfuric acids, for example formic acid, acetic acid, propionic acid, pivalic acid, diethylacetic acid, malonic acid, succinic acid, pimelic acid, fumaric acid, maleic acid, lactic acid, tartaric acid, malic acid, citric acid, gluconic acid, ascorbic acid, nicotinic acid, isonicotinic acid, methane- or ethanesulfonic acid, ethanedisulfonic acid, 2-hydroxyethanesulfonic acid, benzenesulfonic acid, p-toluenesulfonic acid, naphthalenemono- and -disulfonic acids, laurylsulfuric acid. Also suitable and preferred in the meaning of the present invention are acidic cationic ion exchanger resins, such as the commercially available Dowex® or Amberlyst® resins. More preference is given to p-toluenesulfonic acid, furthermore hydrochloric acid, methanesulfonic acid, sulfuric acid or camphorsulfonic acid, or acidic cationic ion exchanger resins, for example Dowex® 50, Amberlyst® 15 or Dowex® DR-2030. Most preferably, the reaction is carried out in the presence of p-toluenesulfonic acid or an acidic cationic ion exchanger resin.

A base of the formula (I) can also be converted into the associated acid-addition salt using an acid, for example by reaction of equivalent amounts of the base and acid in an inert solvent, such as ethanol, with subsequent evaporation. Particularly suitable acids for this reaction are those which give physiologically acceptable salts. Thus, it is possible to use inorganic acids, for example the aforementioned ones. Salts with physiologically unacceptable acids, for example picrates, can be used for the isolation and/or purification of the compounds of the formula (I).

With regard to that stated above, it can be seen that the expressions “pharmaceutically acceptable salt” and “physiologically acceptable salt”, which are used interchangeable herein, in the present connection are taken to mean an active ingredient which comprises a compound according to the invention in the form of one of its salts, in particular if this salt form imparts improved pharmacokinetic properties on the active ingredient compared with the free form of the active ingredient or any other salt form of the active ingredient used earlier. The pharmaceutically acceptable salt form of the active ingredient can also provide this active ingredient for the first time with a desired pharmacokinetic property which it did not have earlier and can even have a positive influence on the pharmacodynamics of this active ingredient with respect to its therapeutic efficacy in the body.

Furthermore, the active ingredients may be administered alone or in combination with yet other treatments. Another synergistic effect may be achieved by using more than one compound of formula (I) in the pharmaceutical composition. All active ingredients can be used either simultaneously or sequentially.

Pharmaceutical formulations can be adapted for administration via any desired suitable method, for example by oral (including buccal or sublingual), rectal, nasal, topical (including buccal, sublingual or transdermal), vaginal or parenteral (including subcutaneous, intramuscular, intravenous or intradermal) methods. Such formulations can be prepared using all processes known in the pharmaceutical art by, for example, combining the active ingredients with the excipient(s) or adjuvant(s).

The pharmaceutical compounds of the invention is produced in a known way using common solid or liquid carriers, diluents and/or additives and usual adjuvants for pharmaceutical engineering and with an appropriate dosage. The amount of excipient material that is combined with the active ingredient(s) to produce a single dosage form varies depending upon the host treated and the particular mode of administration. Suitable excipients include organic or inorganic substances that are suitable for the different routes of administration, such as enteral (e.g. oral), parenteral or topical application, and which do not react with the active ingredients of the invention or salts thereof. Examples of suitable excipients are water, vegetable oils, benzyl alcohols, alkylene glycols, polyethylene glycols, glycerol triacetate, gelatin, carbohydrates, such as lactose or starch, magnesium stearate, talc, and petroleum jelly.

Pharmaceutical formulations adapted for oral administration can be administered as separate units, such as, for example, capsules or tablets; powders or granules; solutions or suspensions in aqueous or non-aqueous liquids; edible foams or foam foods; or oil-in-water liquid emulsions or water-in-oil liquid emulsions. Thus, for example, in the case of oral administration in the form of a tablet or capsule, the active ingredients can be combined with an oral, non-toxic and pharmaceutically acceptable inert excipient, such as, for example, ethanol, glycerol, water and the like. Powders are prepared by comminuting the active ingredients, especially the compounds, to a suitable fine size and mixing it with a pharmaceutical excipient comminuted in a similar manner, such as, for example, an edible carbohydrate, such as, for example, starch or mannitol. A flavor, preservative, dispersant and dye may likewise be present. Capsules are produced by preparing a powder mixture as described above and filling shaped gelatin shells therewith. Glidants and lubricants, e.g. highly disperse silicic acid, talc, magnesium stearate, calcium stearate or polyethylene glycol in solid form, can be added to the powder mixture before the filling operation. A disintegrant or solubiliser, such as agar-agar, calcium carbonate or sodium carbonate, may likewise be added in order to improve the availability of the medicament after the capsule has been taken.

In addition, if desired or necessary, suitable binders, lubricants and disintegrants as well as dyes can likewise be incorporated into the mixture. Suitable binders include starch, gelatin, natural sugars, such as, for example, glucose or beta-lactose, sweeteners made from maize, natural and synthetic rubber, such as, for example, acacia, tragacanth or sodium alginate, carboxymethylcellulose, polyethylene glycol, waxes, and the like. The lubricants used in these dosage forms include sodium oleate, sodium stearate, magnesium stearate, sodium benzoate, sodium acetate, sodium chloride and the like. The disintegrants include, without being restricted thereto, starch, methylcellulose, agar, bentonite, xanthan gum and the like. The tablets are formulated by, for example, preparing a powder mixture, granulating or dry-pressing the mixture, adding a lubricant and a disintegrant and pressing the entire mixture to give tablets. A powder mixture is prepared by mixing the active ingredients, especially the compounds, comminuted in a suitable manner with a diluent or a base, as described above, and optionally with a binder, such as, for example, carboxymethylcellulose, an alginate, gelatin or polyvinylpyrrolidone, a dissolution retardant, such as, for example, paraffin, an absorption accelerator, such as, for example, a quaternary salt, and/or an absorbent, such as, for example, bentonite, kaolin or dicalcium phosphate. The powder mixture can be granulated by wetting it with a binder, such as, for example, syrup, starch paste, acadia mucilage or solutions of cellulose or polymer materials and pressing it through a sieve. As an alternative to granulation, the powder mixture can be run through a tableting machine, giving lumps of non-uniform shape, which are broken up to form granules. The granules can be lubricated by addition of stearic acid, a stearate salt, talc or mineral oil in order to prevent sticking to the tablet casting moulds. The lubricated mixture is then pressed to give tablets. The active ingredients according to the invention can also be combined with a free-flowing inert excipient and then pressed directly to give tablets without carrying out the granulation or dry-pressing steps. A transparent or opaque protective layer consisting of a shellac sealing layer, a layer of sugar or polymer material and a gloss layer of wax may be present. Dyes can be added to these coatings in order to be able to differentiate between different dosage units.

Oral liquids, such as, for example, solution, syrups and elixirs, can be prepared in the form of dosage units so that a given quantity comprises a pre-specified amount of the active ingredients. Syrups can be prepared by dissolving the active ingredients in an aqueous solution with a suitable flavor, while elixirs are prepared using a non-toxic alcoholic vehicle. Suspensions can be formulated by dispersion of the active ingredients, especially the compounds, in a non-toxic vehicle. Solubilisers and emulsifiers, such as, for example, ethoxylated isostearyl alcohols and polyoxyethylene sorbitol ethers, preservatives, flavor additives, such as, for example, peppermint oil or natural sweeteners or saccharin, or other artificial sweeteners and the like, can likewise be added.

The dosage unit formulations for oral administration can, if desired, be encapsulated in microcapsules. The formulation can also be prepared in such a way that the release is extended or retarded, such as, for example, by coating or embedding of particulate material in polymers, wax and the like.

The compounds according to the invention and salts, solvates and physiologically functional derivatives thereof can be administered in the form of liposome delivery systems, such as small unilamellar vesicles, large unilamellar vesicles and multilamellar vesicles. Liposomes can be formed from various phospholipids, such as cholesterol, stearylamine or phosphatidylcholines.

The compounds according to the invention can also be fused or complexed with another molecule that promotes the directed transport to the destination, the incorporation and/or distribution within the target cells. The compounds according to the invention and the salts, solvates and physiologically functional derivatives thereof can also be delivered using monoclonal antibodies as individual carriers to which the compound molecules are coupled. The compounds can also be coupled to soluble polymers as targeted medicament carriers. Such polymers may encompass polyvinylpyrrolidone, pyran copolymer, polyhydroxypropylmethacrylamidophenol, polyhydroxyethylaspartamidophenol or polyethylene oxide polylysine, substituted by palmitoyl radicals. The compounds may furthermore be coupled to a class of biodegradable polymers which are suitable for achieving controlled release of a medicament, for example polylactic acid, poly-epsilon-caprolactone, polyhydroxybutyric acid, polyorthoesters, polyacetals, polydihydroxypyrans, polycyanoacrylates and cross-linked or amphipathic block copolymers of hydro-gels.

Pharmaceutical formulations adapted for transdermal administration can be administered as independent plasters for extended, close contact with the epidermis of the recipient. Thus, for example, the active ingredients can be delivered from the plaster by iontophoresis, as described in general terms in Pharmaceutical Research, 3(6), 318 (1986).

Pharmaceutical compositions adapted for topical administration can be formulated as ointments, creams, suspensions, lotions, powders, solutions, pastes, gels, sprays, aerosols or oils. For the treatment of the eye or other external tissue, for example mouth and skin, the formulations are preferably applied as topical ointment or cream. In the case of formulation to give an ointment, the active ingredients can be employed either with a paraffinic or a water-miscible cream base. Alternatively, the active ingredients can be formulated to give a cream with an oil-in-water cream base or a water-in-oil base. Pharmaceutical formulations adapted for topical application to the eye include eye drops, in which the active ingredients are dissolved or suspended in a suitable carrier, in particular an aqueous solvent. Pharmaceutical formulations adapted for topical application in the mouth encompass lozenges, pastilles and mouthwashes.

Pharmaceutical formulations adapted for rectal administration can be administered in the form of suppositories or enemas. For the preparation of suppositories, the active principles are mixed in a manner that is known per se with a suitable base constituent, such as polyethylene glycol or semi-synthetic glycerides.

Pharmaceutical formulations adapted for nasal administration in which the carrier substance is a solid comprise a coarse powder having a particle size, for example, in the range 20-500 microns, which is administered in the manner in which snuff is taken, i.e. by rapid inhalation via the nasal passages from a container containing the powder held close to the nose. Suitable formulations for administration as nasal spray or nose drops with a liquid as carrier substance encompass active-ingredient solutions in water or oil. Pharmaceutical formulations adapted for administration by inhalation encompass finely particulate dusts or mists, which can be generated by various types of pressurized dispensers with aerosols, nebulisers or insufflators. Insulin and the compound of formula (I) may be administered by inhalation. The efficacy of inhalation of insulin is especially discussed by Skyler et al. (2001) Lancet 357(9253): 331-335. For example, aerosols and inhalation methods developed by Inhale Therapeutic Systems, CA, and described in U.S. Pat. No. 5,997,848 make it possible to optimize the absorption and the reproducibility of the dose delivered.

Pharmaceutical formulations adapted for vaginal administration can be administered as pessaries, tampons, creams, gels, pastes, foams or spray formulations.

Pharmaceutical formulations adapted for parenteral administration include aqueous and non-aqueous sterile injection solutions comprising antioxidants, buffers, bacteriostatics and solutes, by means of which the formulation is rendered isotonic with the blood of the recipient to be treated; and aqueous and non-aqueous sterile suspensions, which may comprise suspension media and thickeners. The formulations can be administered in single-dose or multi-dose containers, for example sealed ampoules and vials, and stored in freeze-dried (lyophilized) state, so that only the addition of the sterile carrier liquid, for example water for injection purposes, immediately before use is necessary. Injection solutions and suspensions prepared in accordance with the recipe can be prepared from sterile powders, granules and tablets. The composition comprising insulin is preferably intended for parenteral administration, more particularly by injection.

It goes without saying that, in addition to the above particularly mentioned constituents, the formulations may also comprise other agents usual in the art with respect to the particular type of formulation; thus, for example, formulations which are suitable for oral administration may comprise flavors.

A therapeutically effective amount of a compound of the present invention depends on a number of factors, including, for example, the age and weight of the human or animal, the precise disease condition which requires treatment, and its severity, the nature of the formulation and the method of administration, and is ultimately determined by the treating doctor or vet. However, an effective amount of a compound according to the invention is generally in the range from 0.1 to 5000 mg/kg of body weight of the recipient (mammal) per day and particularly typically in the range from 1 to 1000 mg/kg of body weight per day and more particularly in the range from 10 to 800 mg/Kg of body weight par day. Thus, the actual amount per day for an adult mammal weighing 70 kg is usually between 70 and 700 mg, where this amount can be administered as an individual dose per day or usually in a series of part-doses (such as, for example, two, three, four, five or six) per day, so that the total daily dose is the same. An effective amount of a salt or solvate or of a physiologically functional derivative thereof can be determined as the fraction of the effective amount of the compound according to the invention per se. It can be assumed that similar doses are suitable for the treatment of other conditions mentioned above.

In a preferred embodiment of the present invention, the pharmaceutical composition is orally or parenterally administered, more preferably parenterally, most preferably as injection solution for parenteral administration. In particular, the active ingredients are provided in a water-soluble form, such as pharmaceutically acceptable salts, which is meant to include both acid and base addition salts. Furthermore, the active ingredients of the invention and salts thereof may be lyophilized and the resulting lyophilizates used, for example, to produce preparations for injection. The preparations indicated may be sterilized and/or may comprise auxiliaries, such as carrier proteins (e.g. serum albumin), lubricants, preservatives, stabilizers, fillers, chelating agents, antioxidants, solvents, bonding agents, suspending agents, wetting agents, emulsifiers, salts (for influencing the osmotic pressure), buffer substances, colorants, flavorings and one or more further active substances, for example one or more vitamins. Additives are well known in the art, and they are used in a variety of formulations.

The respective dose or dosage range for administering the pharmaceutical composition according to the invention is sufficiently high in order to achieve the desired prophylactic or therapeutic effect of reducing symptoms of diseases, which are associated with IR, as set forth below. It will be understood that the specific dose level, frequency and period of administration to any particular human will depend upon a variety of factors including the activity of the specific compound employed, the age, body weight, general state of health, gender, diet, time and route of administration, rate of excretion, drug combination and the severity of the particular disease to which the specific therapy is applied. Using well-known means and methods, the exact dose can be determined by one of skill in the art as a matter of routine experimentation.

The terms “effective amount” or “effective dose” or “dose” are interchangeably used herein and denote an amount of the pharmaceutical compound having a prophylactically or therapeutically relevant effect on a disease or pathological conditions, i.e. which causes in a tissue, system, animal or human a biological or medical response which is sought or desired, for example, by a researcher or physician. A “prophylactic effect” reduces the likelihood of developing a disease or even prevents the onset of a disease. A “therapeutically relevant effect” relieves to some extent one or more symptoms of a disease or returns to normality either partially or completely one or more physiological or biochemical parameters associated with or causative of the disease or pathological conditions. In addition, the expression “therapeutically effective amount” denotes an amount which, compared with a corresponding subject who has not received this amount, has the following consequence: improved treatment, healing, prevention or elimination of a disease, syndrome, condition, complaint, disorder or side-effects or also the reduction in the advance of a disease, complaint or disorder. The expression “therapeutically effective amount” also encompasses the amounts which are effective for increasing normal physiological function.

Pharmaceutical formulations can be administered in the form of dosage units which comprise a predetermined amount of active ingredient per dosage unit. The concentration of the prophylactically or therapeutically active ingredient in the formulation may vary from about 0.1 to 100 wt %. Preferably, the compound of formula (I) or the pharmaceutically acceptable salts thereof are administered in doses of approximately 0.5 to 1.000 mg, more preferably between 1 and 500 mg. It is even most preferred that the unit dose of the compound of formula (I) comprises 12.5 to 500 mg of said compound.

All the references cited herein are incorporated by reference in the disclosure of the invention hereby.

It is to be understood that this invention is not limited to the particular methods, specific substances, uses and kits described herein, as such matter may, of course, vary. It is also to be understood that the terminology used herein is for the purpose of describing particular embodiments only and is not intended to limit the scope of the present invention, which is only defined by the appended claims. As used herein, including the appended claims, singular forms of words such as “a,” “an,” and “the” include their corresponding plural referents unless the context clearly dictates otherwise. Thus, e.g., reference to “a salt” includes a single or several different salts and vice versa, and reference to “a method” includes reference to equivalent steps and methods known to a person of ordinary skill in the art, and so forth. Unless otherwise defined, all technical and scientific terms used herein have the same meaning as commonly understood by a person of ordinary skill in the art to which this invention belongs.

Although methods and materials similar or equivalent to those described herein can be used in the practice or testing of the present invention, suitable examples are described below. The following examples are provided by way of illustration and not by way of limitation. Within the examples, standard reagents and buffers that are free from contaminating activities (whenever practical) are used. The examples are particularly to be construed such that they are not limited to the explicitly demonstrated combinations of features, but the exemplified features may be unrestrictedly combined again if the technical problem of the invention is solved.

### Pharmaceutical Preparations

A) Injection vials: A solution of 100 g of one or more active ingredients according to the invention and 5 g of disodium hydrogen phosphate in 3 l of bidistilled water was adjusted to pH 6.5 using 2 N hydrochloric acid, sterile filtered, transferred into injection vials, lyophilized under sterile conditions and sealed under sterile conditions. Each injection vial contained 5 mg of active ingredient(s).

B) Suppositories: A mixture of 20 g of one or more active ingredients according to the invention was melted with 100 g of soya lecithin and 1400 g of cocoa butter, poured into moulds and allowed to cool. Each suppository contained 20 mg of active ingredient(s).

C) Solution: A solution was prepared from 1 g of one or more active ingredients according to the invention, 9.38 g of NaH₂PO₄.2H2O, 28.48 g of Na₂HPO₄.12H₂O and 0.1 g of benzalkonium chloride in 940 ml of bidistilled water. The pH was adjusted to 6.8, and the solution was made up to 1 l and sterilized by irradiation. This solution could be used in the form of eye drops.

D) Ointment: 500 mg of one or more active ingredients according to the invention were mixed with 99.5 g of Vaseline under aseptic conditions.

E) Tablets: A mixture of 1 kg of one or more active ingredients according to the invention, 4 kg of lactose, 1.2 kg of potato starch, 0.2 kg of talc and 0.1 kg of magnesium stearate was pressed to give tablets in a conventional manner in such a way that each tablet contained 10 mg of active ingredient(s). Other tablets with the active ingredient 5,6-dihydro-4-dimethylamino-2-imino-6-methyl-1,3,5-triazine were prepared according to the Tables 4-9.

F) Coated tablets: Tablets were pressed analogously to the previous paragraph E) and subsequently coated in a conventional manner with a coating of sucrose, potato starch, talc, tragacanth and dye.

G) Capsules: 2 kg of one or more active ingredients according to the invention were introduced into hard gelatin capsules in a conventional manner in such a way that each capsule contained 20 mg of the active ingredient(s).

H) Ampoules: A solution of 1 kg of one or more active ingredients according to the invention in 60 l of bidistilled water was sterile filtered, transferred into ampoules, lyophilized under sterile conditions and sealed under sterile conditions. Each ampoule contained 10 mg of active ingredient(s).

I) Inhalation spray: 14 g of one or more active ingredients according to the invention were dissolved in 10 l of isotonic NaCl solution, and the solution was transferred into commercially available spray containers with a pump mechanism. The solution could be sprayed into the mouth or nose. One spray shot (about 0.1 ml) corresponded to a dose of about 0.14 mg.

1. A method of preventing or treating cancer in a subject in need thereof, comprising administering to said subject a therapeutically effective amount of one or more compounds 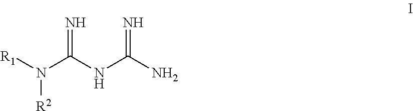
in which
R¹, R² each, independently of one another, denote H, A, Alk, (CH₂)ₙAr, (CH₂)ₙCyc or (CH₂)ₙHet,
R′ and R² together also denote an alkylene chain having 2, 3, 4, 5 or 6 C atoms, in which one CH₂ group may be replaced by O, S, SO, SO₂, NH, NR⁸, NCOR⁸ or NCOOR⁸,
and wherein the alkylene chain is unsubstituted or mono-, di- or trisubstituted by Hal, A, OR¹¹, N(R¹¹)₂, NO₂, CN, phenyl, ═O, CON(R¹¹)₂, NR¹¹COA, NR¹¹CON(R¹¹)₂, NR¹¹SO₂A, COR¹¹, SO₂N(R¹¹)₂, S(O)ₘA, —[C(R¹¹)₂]ₙ—COOR¹¹ and/or —O[C(R¹¹)₂]ₒ—COOR¹¹,
A denotes unbranched or branched alkyl having 1-10 C atoms, in which one, two or three CH and/or CH₂ groups may be replaced by O, S, SO, SO₂, NH, NR⁸ and/or by —CH═CH— groups and/or, in addition, 1-5 H atoms may be replaced by F, Cl, Br and/or R⁷,
Cyc cycloalkyl having 3-7 C atoms,
Alk denotes alkenyl or alkinyl having 2-6 C atoms,
R⁷ denotes COOR⁹, CONR⁹R¹⁰, NR⁹R¹⁰, NHCOR⁹, NHCOOR⁹ or OR⁹,
R⁸ denotes cycloalkyl having 3-7 C atoms, cycloalkylalkylene having 4-10 C atoms, Alk or
unbranched or branched alkyl having 1-6 C atoms,
R⁹, R¹⁰ each, independently of one another, denote H or alkyl having 1-6 C atoms, in which 1-3 CH₂ groups may be replaced by O, S, SO, SO₂, NH, NMe or NEt and/or, in addition, 1-5 H atoms may be replaced by F and/or Cl,
Ar denotes phenyl, naphthyl or biphenyl, each of which is unsubstituted or mono-, di- or trisubstituted by Hal, A, OR¹¹, N(R¹¹)₂, NO₂, CN, phenyl, CON(R¹¹)₂, NR¹¹COA, NR¹¹CON(R¹¹)₂, NR¹¹SO₂A, COR¹¹, SO₂N(R¹¹)₂, S(O)ₘA, —[C(R¹¹)₂]ₙ—COOR¹¹ and/or —O[C(R¹¹)₂]ₒ—COOR¹¹,
Het denotes a mono- or bicyclic saturated, unsaturated or aromatic heterocycle having 1 to 4 N, O and/or S atoms, which may be mono-, di- or trisubstituted by Hal, A, OR¹¹, N(R¹¹)₂, NO₂, CN, COOR¹¹, CON(R¹¹)₂, NR¹¹COA, NR¹¹SO₂A, COR¹¹, SO₂NR¹¹, S(O)ₘA, ═S, ═NR¹¹ and/or ═O (carbonyl oxygen),
R¹¹ denotes H or A,
Hal denotes F, Cl, Br or I,
m denotes 0, 1 or 2,
n denotes 0, 1, 2, 3 or 4,
o denotes 1, 2 or 3,
with the exclusion of the compounds of formula I in which:
a-R¹═H, R²═H;
b-R¹═H, R²=phenethyl;
or a pharmaceutically usable salt, solvate, tautomer or stereoisomer or a mixture thereof, including mixtures thereof.

2. The method of claim 1,
in which
R¹ and R² each, independently of one another, denote H, A, Alk, (CH₂)ₙAr, (CH₂)ₙCyc or (CH₂)ₙHet, and
R¹ and R² together also denote an alkylene chain having 2, 3, 4, 5 or 6 C atoms, in which one CH₂ group may be replaced by O, NH or NR⁸.

3. The method of claim 1,
in which A denotes unbranched or branched alkyl having 1-6 C atoms, in which 1-5 H atoms may be replaced by F, Cl and/or Br.

4. The method of claim 1,
in which Ar denotes phenyl, which is unsubstituted or mono-, di- or trisubstituted by Hal, A, OR¹¹, N(R¹¹)₂, NO₂, CN, phenyl, and/or CON(R¹¹)₂.

5. The method of claim 1,
in which Het denotes a monocyclic saturated, unsaturated or aromatic heterocycle having 1 to 2 N, O and/or S atoms, and which may be mono-, di- or trisubstituted by Hal, A, and/or ═O (carbonyl oxygen).

6. The method of claim 1, in which R¹ and R² each, independently of one another, denote alkyl having 1-4 C atoms.

7. The method of claim 1, in which R¹ and R² denote methyl.

8. The method of claim 1, wherein said cancer is selected from the group consisting of tumors of the squamous epithelium, bladder, stomach, kidneys, head and neck, oesophagus, cervix, thyroid, intestine, liver, brain, prostate, urogenital tract, lymphatic system, stomach, bones, skin, the larynx, and lung.

9. The method of claim 1, wherein said cancer is selected from the group consisting of monocytic leukaemia, lung adenocarcinoma, small-cell lung carcinomas, pancreatic cancer, glioblastomas, breast carcinoma, lung adenocarcinoma, small-cell lung carcinomas, pancreatic cancer, colon carcinoma, bone malignant tumor, chondrosarcoma, Ewing's sarcoma, melanoma.

10. The method of claim 1, in which R¹ and R² denote methyl, and said cancer is melanoma.

11. The method of claim 1, wherein the subject also suffers from a disease selected from diabetes, dyslipidemia, hypertension, retinopathy, nephropathy, neuropathy, and obesity connected with Metabolic Syndrome.

12. The method of claim 11, wherein said disease is diabetes.

13. The method of claim 1,
in which R¹ and R² denote methyl, and where the subject also suffers from diabetes.

14. The method of claim 1 further comprising the step of administering to said subject at least one further medicament or active ingredient.

15. The method of claim 14, wherein said further medicament or active ingredient is an anticancer agent.

16. The method of claim 9, wherein said bone malignant tumor is osteosarcoma
